(19) United States
(12) Patent Application Publication
(10) Pub. No.: US 2004/0010315 A1
(43) Pub. Date: Jan. 15, 2004

(54) Self-expanding intervertebral device

(76) Inventor: John K. Song, Nashville, TN (US)

Correspondence Address: John K. Song, 517 Fairfax Avenue, Nashville, TN, 37212, US

(21) Appl. No.: 10/400,610

(22) Filed: Mar. 27, 2003

Related U.S. Application Data
(60) Provisional application No. 60/368,915, filed on Mar. 29, 2002.

(51) Int. Cl. A61F 2/44

(52) U.S. Cl. 623/17.16; 623/1.18; 623/23.53

(57) ABSTRACT

A design for a self-expanding intervertebral device is described for use in spinal stabilization. The device is designed to be collapsed into a smaller form for ease of implantation and then re-expand to a larger, final size in order to first distract and then maintain this distraction rigidly once implanted. In all forms of the device, the key design element comprises angled or rounded internal structural members to allow ease of initial deformation. Other key features include no mechanically moving parts, implant is a single piece device, and the device contains a cavity for placement of suitable additional material to augment structural strength or promote bone fusion. The device design is applicable to devices designed for placement between adjacent vertebral endplates or longer (taller) constructs spanning non-adjacent endplates.

### CROSS-REFERENCE TO RELATED APPLICATIONS

This application is entitled to the benefit of Provisional Patent Application Serial No. 60/268,915 filed Mar. 29,2002.

Federally Sponsored Research: Not Applicable

Sequence Listing/Program: Not Applicable

### BACKGROUND OF THE INVENTION

The present invention concerns devices implanted in the spinal column for the purpose of stabilizing/fusing the spine. The invention describes a design for an intervertebral device which can be collapsed for ease of implantation and then will self-expand once implanted and a stimulus is given to the device.

The spinal column is made up of bones (vertebrae) stacked one upon the other and separated by cushions (discs). The top and bottom faces of the vertebral bodies are called endplates and the disc material (comprising a tough, fibrous outer lining called the annulus and a softer, inner substance called the nucleus) is contained between the endplates. In patients requiring spinal fusion, the disc material is often removed and an intervertebral device is placed between the exposed endplates to maintain the height of the intervertebral space. Often, a substance is placed into a cavity of the intervertebral device to promote bone fusion between the endplates to stabilize the spine.

If only the disc space is removed (discectomy) and an intervertebral device placed, the device is often called an interbody device This may be performed at multiple disc spaces (called levels) and multiple devices may be placed at each level. These interbody devices may be placed from a variety of approaches. Examples include a lumbar interbody device placed from a posterior approach (LIF), an lumbar interbody device placed from an anterior abdominal approach (ALIF), and one placed from a lateral/oblique approach past the transverse process of the vertebra (TLIF). These devices are not limited to the lumbar spine but may be placed in any disc space within the spinal column.

In some cases, removal of the actual body of one or more vertebra may be required (in a procedure called a corpectomy). In these instances, it is imperative that a rigid structural member replace the removed body/bodies. In this instance, the intervertebral device spans more than one disc space but still only abuts one endplate above and one endplate below.

In many cases, an appropriately selected intervertebal device may be too large to be placed safely where desired. From a posterior approach, hazards include the dura, spinal cord, and nerve roots. Harm to these structures can lead to patient morbidity and even paralysis. In addition, large implants may be able to be placed but only with some difficulty and force.

Thus, there is a need for an interbody device which is implanted at one, smaller size but then can re-expand to a final desired size once in place.

Many inventors have described designs for expanding intervertebral devices. However, most are either devices consisting of multiple pieces joined together with hinges and pivots (U.S. Pat. No. 6,193,757 and U.S. 6,183,517 and U.S. 6,102,950 and U.S. 6,126,689) or are deformable devices that require the application of an external force to either hold the device in the collapsed state like a spring (U.S. Pat. No. 5,749,916) or to re-expand the device to its original state (U.S. Pat. No. 5,059,193). The current invention is a single-component device which does not require the continued application of force to constrain the device in the collapsed state and does not require the application of force to re-expand the device.

Foley (U.S. Pat. No. 6,193,757) describes an embodiment where the lateral walls or “arms” are deformable and possibly made of a shape-memory alloy. However, no specific design feature, such as the rounded or “V” shaped struts of the current invention, are described to assist with deformation.

Richelsoph (U.S. Pat. No. 5,749,916) describes an implant which can slightly “give” under axial loading. The author of die current invention feels that movement can lead to failure of bone fusion (called a pseudoarthosis) and thus the current invention is designed to be rigid after implantation.

Kuslich (U.S. Pat. No. 5,059,193) describes a device with multiple ribs which are designed to deform under a deforming force from a first, smaller, implant size to a larger second size. The current invention is designed to collapse under a deforming load and does not require a deforming force to achieve the larger implant size.

Similar appearing devices have also been described for use in endovascular procedures and are commonly called stents. Most describe devices specifically for use in an intravascular setting (U.S. Pat. No. 5,019,090).

Pinchuk (U.S. Pat. No. 5,019,090) describes a continuous, helical circumferential sections of a deformable device. This is markedly different from the current invention.

Hess (U.S. Pat. No. 5,197,978) describes a tissue-supporting device which is made of nitinol and is able to be inserted into the patient in its undeformed martensite state, expanded in its martensite state and then potentially collapsible by heating above its austenite transformation temperature to return to its original size. The current device is intended to be implanted in a deformed martensite state and re-expanded in its austentite state. The strength properties of nitinol are superior in its austenite state. Shanley (U.S. Pat. No. 6,293,967 and U.S. 6,241,762) describes a device containing multiple ductile hinges that is radially expandable. However, the device requires the application of a force to expand the device to cause the ductile hinges to experience plastic deformation. The deformable members of the current invention do not undergo plastic deformation under expansion, rather the nitinol experiences a unique change in crystalline structure to return to its original shape.

Limon (U.S. Pat. No. 6,273,910) describes “U” shaped structures linked together in a serpentine pattern that can expand in response to an outwardly directed radial force. Again, the current invention does not require the application of force to expand.

### SUMMARY OF THE INVENTION

The present invention describes a design for intervertebral devices which allows the device to be collapsed to a smaller insertion size from the larger, rigid, final implant size. The design allows the device to minimize the stresses imparted to the device by the act of collapsing it without allowing it to exceed its yield strength and break. As currently conceived, the device is made of a shape memory substance (e.g. nitinol) although future advances may allow it to be made of some other material such as a plastic or polymer.

In all embodiments of the design, “V” shaped or rounded structural members designed to facilitate deformation under certain conditions allow collapse without breakage and with minimal force compared to internal structural members not designed for deformation. The device design includes a central cavity for containment of bone graft, bone cement or other implant material.

Multiple applications of the design are given in this application along with multiple embodiments. It is important to note that the current invention consists of a single component and requires no mechanical intervention on the part of the user to re-expand except for application of a stimulus such as heat to initiate the re-expansion. Also, it is important to note that the device is NOT a spring and does not require containment to keep it in its collapsed insertion size and does not store energy in the same manner that a spring does when deformed.

In one embodiment of the design, the device is designed for placement between adjacent endplates—possible embodiments include a cylindrical device or a multi-faceted possibly box-like structure incorporating the angled or rounded internal structural members that are capable of allowing the device to be collapsed in all directions.

In another application of the design, the device is designed to span non-adjacent endplates and again, embodiments include a cylindrical or a box-like device with the described internal members. The main advantage in this case is expansion in the vertical dimension although a device that was collapsible in the device's axial dimension could be devised.

The devices described above are currently made of a shape memory alloys (e.g. nitinol). Nitinol is, in its most common form, a binary alloy and has the interesting property of being able to regain a “set” shape from a deformed shape under certain conditions in a manner not similar to springs or elastic structures.

In order to create the devices of the current invention, nitinol is machined into the appropriate and desired shape. Currently available manufacturing technologies include wire or ram electrical discharge machining (EDM), laser cutting, and abrasive or conventional machining techniques. Following that, the device must have its shape “set”. If the device is machined in its smaller, implant size (as are many vascular stents), it must be expanded to its final size and then “set”. If the device is created in its final size, it is then ready to be “set”.

In order to set the shape of the device, the part must be heat-treated to approximately 400-700° C. for several minutes. It is then rapidly cooled—this process leaves an oxide layer on the device which may be removed through any number of well-known finishing processes.

In order to collapse the device into its smaller, implantation size, the device must be cooled below its martensite transformation temperature. This is easily performed using dry ice or liquid nitrogen or even cold water depending on the transformation temperature. The device is then deformed to the desired, smaller implantation size. From this point, the device must be kept below its austenite transformation temperature.

The device is provided in a sterile, collapsed condition to the surgeon. Sterilization can occur at any point after heat treatment and the device may be collapsed anywhere between heat treatment and the operating room.

Any number of methods may be used to collapse the device from a simple vise to custom made jigs and collets depending on the shape and size of the implant.

The device may be implanted using tools that grip the outer or inner surface of the device. The surgical procedure for implanting the device varies depending on the size of the device. For an intervertebral device designed to span adjacent endplates (commonly called an interbody device), the intervertebral disk space can be approached from a posterior, anterior, or lateral/transverse direction. A hole is created in the annulus of the disk and disk material is removed. Preparation of the endplates by scraping or curetting is usually required to enhance bone fusion. The device is then inserted into the space created. Expansion of the device then occurs when an appropriate stimulus is applied (heat, electricity, etc.). The central cavity of the device may be filled at this time.

For a multiple-level intervertebral device spanning non-adjacent endplates, an anterior or lateral approach is usually used. Posterior approaches are usually not feasible due to the larger sizes of these devices and the spinal cord, dura, and nerves that are between the surgeon and the vertebra. Well-known techniques for corpectomies and endplate preparation are performed and the multi-level device is inserted in its collapsed state. Appropriate stimulus is applied and the device expands to engage the .endplates. The central cavity of the device may be filled at this time.

### BRIEF DESCRIPTION OF THE DRAWINGS

FIG. 1(a). End view of cylindrical implant with many “V”-shaped collapsible members.

FIG. 1(b). Side view of cylindrical implant with many “V”-shaped members.

FIG. 1(c). Perspective view of cylindrical implant with many “V”-shaped collapsible members.

FIG. 2(a). End view of cylindrical implant with few “V”-shaped collapsible members.

FIG. 2(b). Side view of cylindrical implant with few “V”-shaped collapsible members.

FIG. 2(c). Perspective view of cylindrical implant with few “V”-shaped collapsible members.

FIG. 3(a). End view of lordotic cylindrical implant with “V”-shaped collapsible members.

FIG. 3(b). Side view of lordotic cylindrical implant with “V”-shaped collapsible members.

FIG. 3(c). Perspective view of lordotic cylindrical implant with “V”-shaped collapsible members.

FIG. 4(a). End view of multi-faceted implant with “V”-shaped collapsible members.

FIG. 4(b). Side view of multi-faceted implant with “V”-shaped collapsible members.

FIG. 4(c). Perspective view of multi-faceted implant with “V”-shaped collapsible members.

FIG. 5(a). End view of multi-faceted implant with “V”-shaped collapsible members.

FIG. 5(b). Side view of multi-faceted implant with “V”-shaped collapsible members.

FIG. 5(c). Perspective view of multi-faceted implant with “V”-shaped collapsible members.

FIG. 6(a). End view of multi-faceted implant with “V”-shaped collapsible members.

FIG. 6(b). Side view of multi-faceted implant with “V”-shaped collapsible members.

FIG. 6(c). Perspective view of multi-faceted implant with “V”-shaped collapsible members.

FIG. 7(a). End view of multi-faceted implant with rounded and “V”-shaped collapsible members.

FIG. 7(b). Side view of multi-faceted implant with rounded and “V”-shaped collapsible members.

FIG. 7(c). Perspective view of multi-faceted implant with rounded and “V”-shaped collapsible members.

FIG. 8(a). Side view of collapsed implant and collapsed disk space.

FIG. 8(b). Side view of expanded implant and expanded disk space.

FIG. 9. Perspective view of a collapsed implant on an applier and an expanded implant.

### DETAILED DESCRIPTION OF THE DRAWINGS

For the purposes of promoting an understanding of the principles of the invention, references will now be made to the embodiments illustrated in the drawings and specific language will be used to describe the same. It will nevertheless be understood that no limitation of scope of the invention is thereby intended, any alterations and further modifications of the illustrated devices, and any further applications of the principles of the invention as illustrated therein being contemplated as would normally occur to one skilled in the art to which the invention relates.

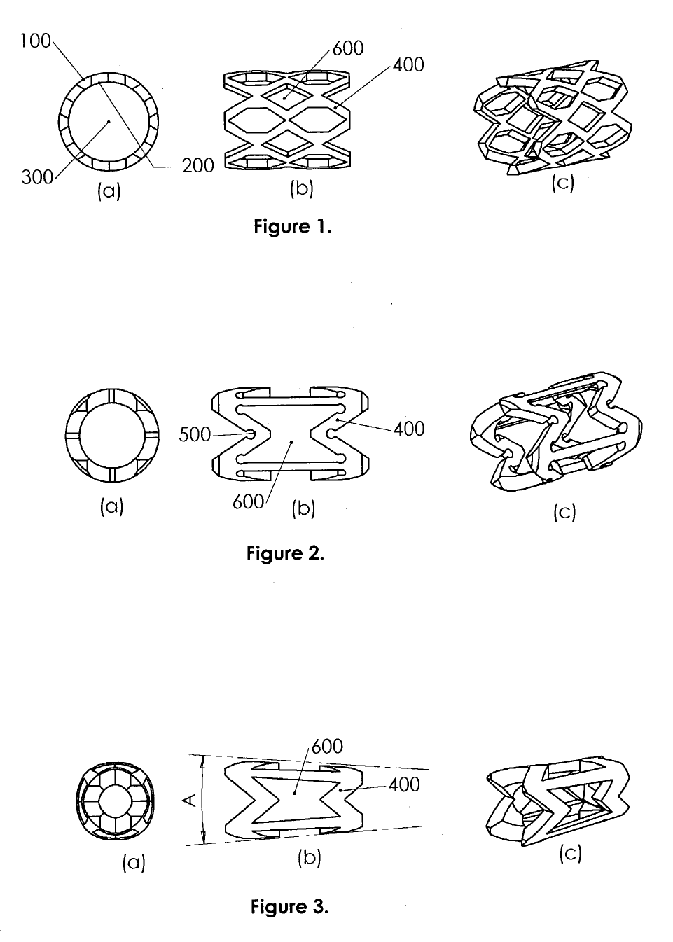

The first 3 (FIGS. 1,2,3)—are a variation of a roughly cylindrical embodiment of the present invention. FIG. 1 illustrates such a device which has multiple “V”-shaped struts which, when the device is cooled below its martensite transformation temperature, allow the device to be collapsed down to a smaller cylinder. Item 100 indicates the outer surface of the device, 200 the inner surface and 300 denotes the hollow central cavity. Item 400 refers to the “V”-shaped deformable strut which allows the device to be easily collapsed. Note that all similar features have not been labeled for clarity of the drawing but are understood to function in a similar manner. FIG. 1(a) illustrates what one would see if the device were implanted in the intervertebral space and one were inspecting the device from the patient's front. FIG. 1(b) illustrates what the device appears as from the patient's side. Openings in the wall of the device 600 allow bone growth from the central cavity 300 to the vertebral endplate which is engaging the outer surface of the device 100.

FIG. 2 is a similar device however with larger but fewer deformable “V”-struts. In addition, there is the addition of stress relief cutouts (Item 500) at the apex of the deformable strut—this feature allows more stress to be applied to the device to collapse it without breaking it. This feature can be incorporated into any of the described embodiments. FIG. 3 illustrates a similar cylindrical device with angle A which provides for spinal lordosis or kyphosis.

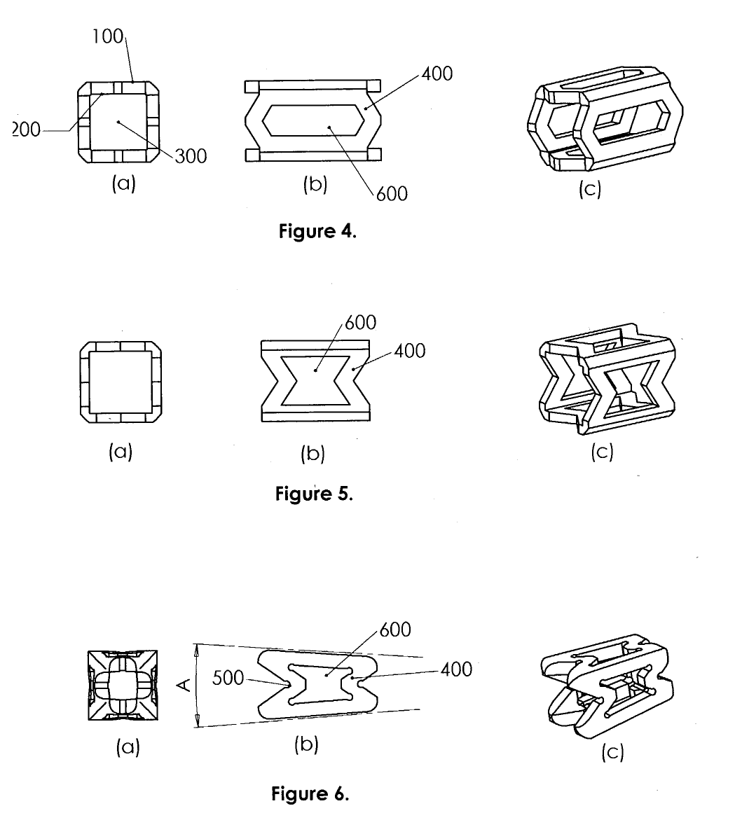

FIGS. 4,5,6 are various embodiments of a multifaceted device. In the embodiments depicted, the device is roughly box-shaped. FIG. 4 depicts deformable “V”struts 400 which are angled away from each other while FIG. 5 depicts “V”struts 400 which are angled towards each other. FIG. 6 shows a lordotic, box-spaced device which has “V”-shaped deformable struts (400) and stress-relief cutouts (item 500). The stress-relief cutouts allow for greater stresses to be imparted to the device without causing the device to break at the apex of the “V”. Again, the lordotic angle is designated by the angle A.

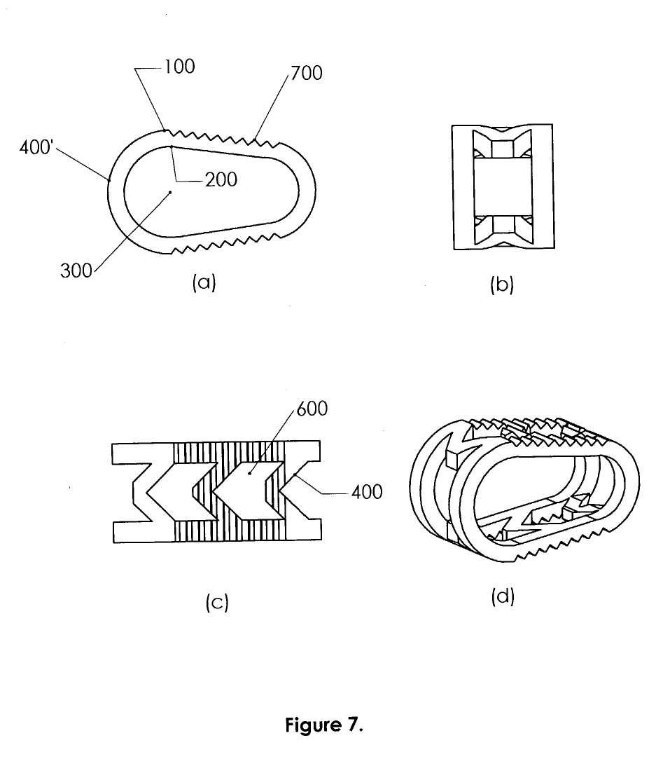

FIG. 7 shows a non-cylindrical implant which has rounded deformable struts (400′) addition to “V”-shaped deformable struts (400). This device incorporates a raised surface detail 700 to improve engagement between the outer surface of the device and the vertebral endplate.

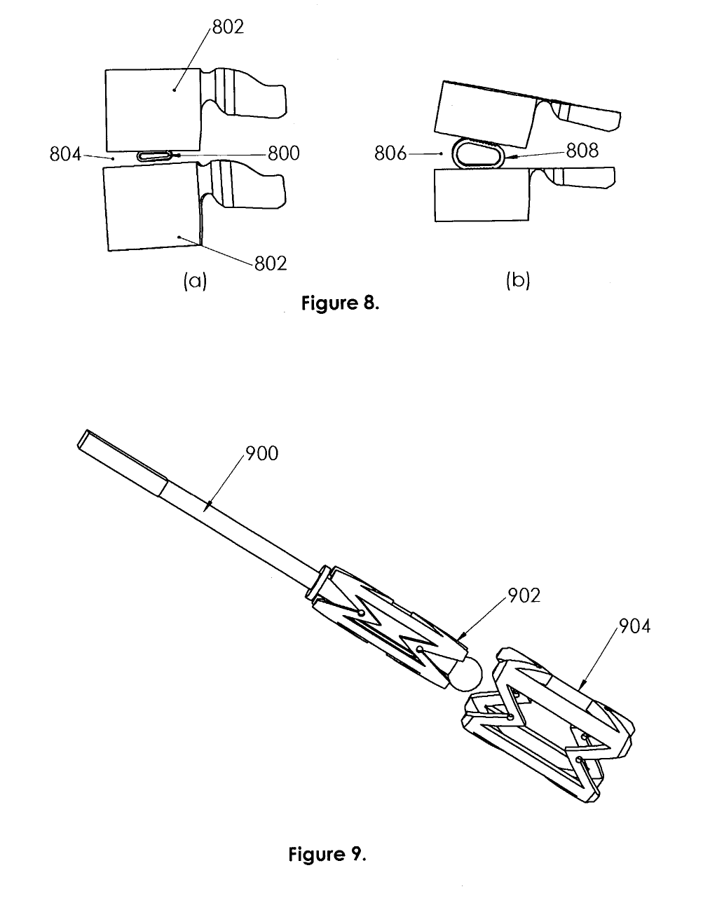

FIG. 8 shows a side view of the invention 800 and adjacent vertebral bodies 802 both with a collapsed intervertebral space 804 (FIG. 8a) and an expanded intervertebral space 806 with an expanded implant 808 (FIG. 8b).

FIG. 9 illustrates what a collapsed cylindrical implant 902 on an application instrument 900 might appear as. An expanded implant 904 is also depicted.

1. A single-component device made from a shape memory material for implanting between spinal vertebral endplates resulting in a rigid construct comprising an inner and outer surface defining the walls of said device, and said walls being continuous and containing rigid and distinct structural members which facilitate deformation of said device into a collapsed state until the application of an external, non-forceful stimulus whereby the natural tendencies of the shape memory material restores said device to the original expanded state in a non-resilient and non-springlike manner.

2. Device of claim 1 having raised details on the outer surface to engage the vertebral endplates.

3. Device of claim 1 being cylindrical in shape.

4. Device of claim 1 having multiple sides.

5. Device of claim 1 having a hollow central cavity contained within the inner surface.

6. Device of claim 1 whereby the structural members are angled or rounded in shape.
i. Device of claim e whereby the structural members contain stress-relief cutouts.

7. Device of claim 1 where the use of external force or device to maintain the collapsed state once deformed is not needed.

8. Device of claim 1 whereby the release of external force or device is not needed to restore said device to the original expanded state.

9. Device of claim 1 wherein the walls of said device contain openings for bone growth to bridge between the hollow central cavity and the vertebral endplates.

10. Device of claim 1 able to distract adjacent or non-adjacent vertebral endplates.

11. Device of claim 1 where the external stimulus is heat energy.

12. A single-component, generally cylindrical device for distracting opposing bony surfaces able to be implanted in the body in a smaller, collapsed size and expanded to a larger, final size, said device comprised of an inner and outer surface defining the walls of the device and said walls being continuous and containing distinct and rigid “V”-shaped structural members which facilitate the deformation of the device to the smaller, collapsed size and said device being made of a shape memory material which is able to restore said device to the larger, final size in a non-resilient and non-springlike manner upon application of an external, non-forceful stimulus.

13. Device of claim 1 having raised details on the outer surface to engage the opposing bony surfaces.

14. Device of claim 1 having a hollow central cavity contained within the inner surface.

15. Device of claim 1 where the use of external force or device to maintain the collapsed state once deformed is not needed.

16. Device of claim 1 whereby the release of external force or device is not needed to restore said device to the original expanded state.

17. Device of claim 1 wherein the walls of said device contain openings for bone growth to bridge between the hollow central cavity and the opposing bony surfaces.

18. Device of claim 1 where the external stimulus is heat energy.

19. A single-component, generally cylindrical device for distracting opposing bony surfaces able to be implanted in the body in a smaller, collapsed size and expanded to a larger, final size, said device comprised of an inner and outer surface defining the walls of the device and said walls being continuous and containing distinct and rigid deformable members forming roughly “V”-shaped structural members with stress-relief cutouts at the apex which facilitate the deformation of the device to the smaller, collapsed size and said device being made of a shape memory material which is able to restore said device to the larger, final size in a non-resilient and non-springlike manner upon application of an external, non-forceful stimulus.

20. Device of claim 1 having raised details on the outer surface to engage the opposing bony surfaces.

21. Device of claim 1 having a hollow central cavity contained within the inner surface.

22. Device of claim 1 where the use of external force or device to maintain the collapsed state once deformed is not needed.

23. Device of claim 1 whereby the release of external force or device is not needed to restore said device to the original expanded state.

24. Device of claim 1 wherein the walls of said device contain openings for bone growth to bridge between the hollow central cavity and the opposing bony surfaces.

25. Device of claim 1 where the external stimulus is heat energy.

26. A single-component, generally cylindrical device for distracting opposing bony surfaces able to be implanted in the body in a smaller, collapsed size and expanded to a larger, final size, said device comprised of an inner and outer surface defining the walls of the device and said walls being continuous and containing distinct and rigid rounded structural members which facilitate the deformation of the device to the smaller, collapsed size and said device being made of a shape memory material which is able to restore said device to the larger, final size in a non-resilient and non-springlike manner upon application of an external, non-forceful stimulus.

27. Device of claim 1 having a hollow central cavity contained within the inner surface.

28. Device of claim 1 having a hollow central cavity contained within the inner surface.

29. Device of claim 1 where the use of external force or device to maintain the collapsed state once deformed is not needed.

30. Device of claim 1 whereby the release of external force or device is not needed to restore said device to the original expanded state.

31. Device of claim 1 wherein the walls of said device contain openings for bone growth to bridge between the hollow central cavity and the opposing bony surfaces.

32. Device of claim 1 where the external stimulus is heat energy.

33. A single-component, non-cylindrical, multi-faceted device for distracting opposing bony surfaces able to be implanted in the body in a smaller, collapsed size and expanded to a larger, final size, said device comprised of an inner and outer surface defining the walls of the device and said walls being continuous and containing distinct and rigid “V”-shaped structural members which facilitate the deformation of the device to the smaller, collapsed size and said device being made of a shape memory material which is able to restore said device to the larger, final size in a non-resilient and non-springlike manner upon application of an external, non-forceful stimulus.

34. Device of claim 1 having raised details on the outer surface to engage the opposing bony sur faces.

35. Device of claim 1 having a hollow central cavity contained within the inner surface.

36. Device of claim 1 where the use of external force or device to maintain the collapsed state once deformed is not needed.

37. Device of claim 1 whereby the release of external force or device is not needed to restore said device to the original expanded state.

38. Device of claim 1 wherein the walls of said device contain openings for bone growth to bridge between the hollow central cavity and the opposing bony surfaces.

39. Device of claim 1 where the external stimulus is heat energy.

40. A single-component, non-cylindrical, multi-faceted device for distracting opposing bony surfaces able to be implanted in the body in a smaller, collapsed size and expanded to a larger, final size, said device comprised of an inner and outer surface defining the walls of the device and said walls being continuous and containing distinct and rigid deformable members forming roughly “V”-shaped structural members with stress-relief cutouts at the apex which facilitate the deformation of the device to the smaller, collapsed size and said device being made of a shape memory material which is able to restore said device to the larger, final size in a non-resilient and non-springlike manner upon application of an external, non-forceful stimulus.

41. Device of claim 1 having raised details on the outer surface to engage the opposing bony surfaces.

42. Device of claim 1 having a hollow central cavity contained within the inner surface.

43. Device of claim 1 where the use of external force or device to maintain the collapsed state once deformed is not needed.

44. Device of claim 1 whereby the release of external force or device is not needed to restore said device to the original expanded state.
